(19) United States
(12) Patent Application Publication
(10) Pub. No.: US 2023/0212106 A1
(43) Pub. Date: Jul. 6, 2023

(54) Facile Synthesis of Aluminum Salts Using Activated Aluminum Precursor

(71) Applicant: Massachusetts Institute of Technology, Cambridge, MA (US)

(72) Inventors: Jude Aaron Kelley, Bolton, MA (US); Alla Ostrinskaya, Needham, MA (US); Eric Richard Morgan, Bolton, MA (US); Roderick Russell Kunz, Acton, MA (US)

(21) Appl. No.: 17/978,010

(22) Filed: Oct. 31, 2022

Related U.S. Application Data
(60) Provisional application No. 63/295,578, filed on Dec. 31, 2021.

(51) Int. Cl. C07C 51/41; C01F 7/66; C01F 7/58; C01B 25/36; C01B 3/08; C07C 59/265; C07C 55/07

(52) U.S. Cl. CPC C07C 51/41; C01F 7/66; C01F 7/58; C01B 25/36; C01B 3/08; C07C 59/265; C07C 55/07

(57) ABSTRACT

The present disclosure provides a method of producing an aluminum salt, comprising reacting activated aluminum metal (Al⁽⁰⁾) with an anion donor. Also provided are aluminum salts prepared by the disclosed methods.

### CROSS REFERENCE TO RELATED APPLICATIONS

This application claims priority to U.S. Provisional Application No. 63/295,578, filed Dec. 31, 2021, the contents of which are herein incorporated by reference in their entirety.

### FEDERAL FUNDING STATEMENT

This invention was made with government support under FA8702-15-D-0001 awarded by the U.S. Air Force. The government has certain rights in the invention.

### BACKGROUND

Aluminum salts have a formula wherein the aluminum is the positively charged cation, and these aluminum cations are combined in some ratio with a negatively charged anion or anions. Mixed salts are also possible, where more than one type of cation or anion exists in the formula of the salt.

Aluminum salts are useful in a variety of applications, ranging from consumer products to industrial use. Aluminum citrate is a valuable salt that is used as a cross-linking agent in the formation of polymers and gels. These gels are often used for oil recovery in commercial oil drilling operations. Aluminum salts such as aluminum chlorohydrate and others are used in many antiperspirants and deodorants.

Aluminum phosphate is commonly used as an adjuvant in human vaccines. Reaction of activated aluminum with phosphoric acid should produce aluminum phosphate as the majority soluble/condensed phase reaction product. U.S. Pat. No. 8,540,955 discloses methods of producing aluminum phosphate starting with aluminum chloride (salt) and a solution of sodium phosphate (tribasic).

Aluminum sulfate is used as a coagulating agent for purification of drinking water, wastewater treatment and in paper production. Typical synthesis of aluminum sulfate involves combination of caustic sulfuric acid with aluminum containing minerals.

Most traditional synthetic preparations of aluminum salts start with a solution of a different aluminum salt than the one ultimately desired. In some cases, this first salt must be independently synthesized prior to the intended reaction. Once the first salt has been procured, it is dissolved in solution and combined with a second salt, base, or acid to create the desired aluminum salt by metathesis (in this case, swapping the anion on the precursor aluminum salt for a different anion desired in the final product).

Accordingly, there exists a need in the art for generalizable and simplified methods of synthesizing aluminum-containing salts in a more cost effective, safe, and efficient manner.

### SUMMARY OF THE INVENTION

This Summary introduces a selection of concepts in simplified form that are described further below in the Detailed Description. This Summary neither identifies key or essential features, nor limits the scope, of the claimed subject matter.

In one aspect, the present disclosure provides a method for preparing an aluminum salt comprising reacting activated aluminum metal (Al⁽⁰⁾) with an anion donor in an aqueous solution; producing an aluminum salt and one or more byproducts of the reaction; and optionally separating the aluminum salt from the byproducts.

In another aspect, the present disclosure provides an aluminum salt prepared by the methods disclosed herein.

### BRIEF DESCRIPTION OF THE DRAWINGS

FIG. 1 shows the negative ion mode Q1 spectrum for aluminum citrate (the black dashed spectrum is aluminum citrate standard, the black solid spectrum is aluminum citrate prepared according to the methods disclosed herein, and the grey spectrum is methanol/water solvent).

FIG. 2 shows the product ion mass spectrometry for aluminum citrate.

FIG. 3 shows the product ion spectrum of m/z 233 for aluminum citrate.

FIGS. 4A-4O show the product ion spectra for aluminum citrate: 354 (4A), 343 (4B), 332 (4C), 317 (4D), 275 (4E), 273 (4F), 255 (4G), 233 (4H), 228 (4I), 215 (4J), 203 (4K), 181 (4L), 151 (4M), 135 (4N), 113 (4O).

FIG. 5 shows the negative ion mode Q1 spectrum for aluminum oxalate (the black dashed spectrum is aluminum oxalate standard, the black solid spectrum is aluminum oxalate prepared according to the methods disclosed herein).

### DETAILED DESCRIPTION

As disclosed herein, the inventors have discovered a method for preparing aluminum salts that overcomes many of the limitations of prior methods.

Traditional chemical synthesis of aluminum salts can be a cumbersome process. For example, two typical routes used to synthesize aluminum citrate, an aluminum salt used as a crosslinker and valued in the field of oil drilling, are as follows:

Al₂(SO₄)₃ (aq)+2 H₃(C₆H₅O₇) (aq)↔2 Al(C₆H₅O₇)(aq)+3 H₂SO₄(aq) (Reaction 1)

and

Al₂(SO₄)₃ (aq)+2 Na₃(C₆H₅O₇) (aq)↔2 Al(C₆H₅O₇)(aq)+3 Na₂SO₄(aq) (Reaction 2)

The metathesis reactions above (described in, e.g., British Patent No. 1,598,709) both require a solution of an aluminum salt to start (aluminum sulfate, Al₂(SO₄)₃ (aq)) and produce not only the desired aluminum citrate (Al(C₆H₅O₇)(aq)) salt, but three equivalents of either a strong acid (sulfuric acid) as in Reaction 1, or a soluble salt (sodium sulfate) as in Reaction 2. These byproducts may not be desired and must be separated/purified from the end reaction mixture. Furthermore, base may need to be added in order to achieve a desirable reaction pH in the case of Reaction 1. Other known methods of preparing aluminum citrate (see, e.g., U.S. Pat. No. 5,559,263) also start with an aluminum salt, namely a solution of aluminum chloride, and produce aluminum citrate via reaction with a citric acid solution. Another known method generates aluminum citrate by reaction of sodium aluminate solutions with citric acid solutions (see, e.g. U.S. Pat. No. 4,560,783).

With the methods disclosed herein, it is possible to start with just activated aluminum metal, i.e. Al⁽⁰⁾ and directly synthesize a desired aluminum salt by immersing the activated aluminum metal in a solution containing only the desired anion donor (i.e. salt, acid or base or combination thereof) for the final salt product. Without wishing to be bound by any particular theory, this is because activated aluminum has the unique property of directly presenting Al⁽⁰⁾ (i.e. elemental aluminum) to solution, which does not typically happen with normal aluminum metal (which rapidly self-passivates by the formation of a self-healing and rapidly formed inert oxide layer (i.e. Al₂O₃)). Activated aluminum is believed to have a disrupted oxide layer that makes Al⁽⁰⁾ continually available to chemical reactions as a reactive precursor. And while there are alternative methods for presenting Al⁽⁰⁾ to solution—e.g. the use of nanoscale aluminum (under an inert atmosphere)—the pyrophoric nature of nanoscale aluminum makes these methods impractical and dangerous.

In accordance with the methods disclosed herein, a more desirable and simplified synthetic route is as shown in exemplary reaction 3:

Al⁽⁰⁾(s)+H₃(C₆H₅O₇) (aq)↔Al(C₆H₅O₇)(aq)+3/2 H₂(g) (Reaction 3)

In the case of Reaction 3 above, solid or slurried activated aluminum metal (Al⁽⁰⁾) alone (i.e. not an aluminum salt or a solution of a salt) reacts with citric acid to form aluminum citrate (the desired product), and one and a half equivalents of hydrogen gas byproduct, which bubbles out of solution. (The hydrogen gas can be collected and used on its own, or safely vented to the atmosphere.) Thus, separation of undesired byproducts is not required with Reaction 3.

Furthermore, the pH of the solution resulting from Reaction 3 trends towards neutral, as the acidic protons from citric acid are reduced by electrons supplied by the activated aluminum:

Al⁽⁰⁾↔Al⁽⁺³⁾+3 e−

3H⁽⁺⁾+3e−↔3/2 H₂(g)

Thus, the concern with acidic pH excursion (as may exist with Reaction 1) is absent. Notably, the methods disclosed herein (exemplified by Reaction 3), using activated aluminum as a reactive precursor, do not require a preexisting aluminum salt solution to synthesize a new aluminum salt, whereas prior methods require a preexisting aluminum salt solution to synthesize a new aluminum salt.

Accordingly, the present disclosure provides a method for preparing an aluminum salt comprising reacting activated aluminum metal (Al⁽⁰⁾) with an anion donor in an aqueous solution; producing an aluminum salt and one or more byproducts of the reaction; and optionally separating the aluminum salt from the byproducts.

As used herein “activated aluminum metal,” or “activated aluminum,” i.e. Al⁽⁰⁾), refer to aluminum metal infused with small quantities of activating materials/metals (e.g. gallium and indium).

As used herein, the term “anion donor” refers to a chemical entity capable of providing an anion for pairing with aluminum in order to form an aluminum salt. The anion donor may be a soluble acid, base, or salt. The term “anion donor” is meant to encompass a single chemical entity or a mixture thereof, i.e. one or more chemical entities in combination.

In the methods disclosed herein, the rapid oxidation of Al⁽⁰⁾ when it reaches aqueous solution provides a steady source of aluminum cations, which rapidly combine with the anions of the anion donor (as presented in solution).

In certain embodiments, the activated aluminum metal is solid or slurried activated aluminum metal.

In certain embodiments, the anion donor is a soluble acid. In certain embodiments, the soluble acid may be citric acid, oxalic acid, nitric acid, sulfuric acid, phosphoric acid, hydrogen chloride, or hydrogen fluoride. Other acids that may be used with the methods disclosed herein include those within the purview of one of skill in the art to form suitable aluminum salts, and are included within the scope of the disclosed methods even where not specifically recited herein.

In certain embodiments, the Al⁽⁰⁾ is reacted with the appropriate soluble anion donor to form aluminum oxalate, aluminum nitrate, aluminum sulfate, aluminum chloride, aluminum fluoride, and other aluminum organic salts, and aluminum halides.

In certain embodiments, the aluminum salts may be prepared by reacting activated aluminum metal in a buffer solution, however alternative and/or simpler solutions (e.g. adding the activated aluminum to an aqueous or protic solution containing concentrations of dissolved anion donor(s)) may be used to produce the desired aluminum salt product(s). Thus, while buffer solutions represent one option for suitable solutions for the methods disclosed herein, other solutions (e.g. sodium citrate alone or sodium oxalate alone) would produce the same aluminum salt product (e.g. aluminum citrate or aluminum oxalate) may be used with the disclosed methods. Suitable solutions and reaction conditions are within the purview of one of skill in the art.

In certain embodiments in which the anion donor is a soluble acid, the byproduct of the reaction is hydrogen gas (H₂). The hydrogen gas may be allowed to bubble out of solution, where it may can be collected and used, or safely ventilated into the atmosphere. In such embodiments (i.e. the reaction with acids), the reaction is particularly “clean” due to the formation of self-removed hydrogen gas.

In some embodiments, the methods disclosed herein may optionally include a step of separating the aluminum salt from the byproducts of the reaction. As used herein, “separating” means disassociating, removing from solution, and/or purifying the aluminum salt by any suitable means, such as extraction, precipitation or the like.

Separating the aluminum salt may be useful in instances where the methods of the invention are performed with anion donors that are salts or bases, which can produce desired aluminum salts but may require some additional separation/purification steps. In a non-limiting example, a similar reaction to Reaction 3 above could be carried out by reacting activated aluminum with a solution of (tribasic) sodium citrate, resulting in aluminum citrate and an additional sodium hydroxide byproduct.

In a further aspect, the disclosure provides aluminum salts prepared in accordance with the methods described supra.

### EXAMPLES

### Example 1: Synthesis of Aluminum Citrate

To synthesize aluminum citrate from activated aluminum, a 0.25 gram pellet of activated aluminum was dropped into a glass vessel containing 400 mL of a sodium citrate (dihydrate) buffer. The buffer solution was prepared as follows: 320 mL of deionized water was placed in the reaction vessel. 9.708 grams of sodium citrate dihydrate (solid) was added to the solution. 1.343 grams of citric acid (solid) was then added to the solution. The solution was stirred to dissolve the solids, and more deionized water was added to achieve a total liquid volume of 400 mL. Once the sodium citrate buffer solution was prepared, the 0.25 gram pellet of activated aluminum was dropped into the solution. The pellet reacts to form bubbles of hydrogen gas and heat. Aluminum citrate is formed as a soluble product (dissolved in aqueous solution).

### Example 2: Synthesis of Aluminum Oxalate

To synthesize aluminum oxalate from activated aluminum, a 0.25 gram pellet of activated aluminum was dropped into a glass vessel containing 400 mL of a sodium oxalate buffer. The buffer solution was prepared as follows: 320 mL of deionized water was placed in the reaction vessel. 6.088 grams of sodium oxalate (solid) was added to the solution. 4.092 grams of oxalic acid (solid) was then added to the solution. The solution was stirred to dissolve the solids, and more deionized water was added to achieve a total liquid volume of 400 mL. Once the sodium oxalate buffer solution was prepared, the 0.25 gram pellet of activated aluminum was dropped into the solution. The pellet reacts to form bubbles of hydrogen gas and heat. Aluminum oxalate is formed as a soluble product (dissolved in aqueous solution).

### Example 3: Characterization of a Reaction of Activated Aluminum with an Aqueous Citrate Buffer

Activated aluminum was reacted with an aqueous citrate buffer to produce soluble aluminum citrate. An electrospray mass spectrometer (AB-Sciex 4000 Triple Quadrupole Mass Spectrometer) was used to detect aluminum citrate (anions) in the post reaction solution.

First, an electrospray mass spectrum of a methanol/water solution was acquired to determine what ions were present in the chosen electrospray fluid (background measurement).

A standard solution of aluminum citrate was prepared using a commercially high purity aluminum citrate solid, which was dissolved in a methanol/water solution and electrosprayed. Aluminum citrate ions were identified via primary mass spectrometry (Q1 mode) and by identification of fragments via product ion scans (as previously reported by Hotta, H. et al., Anal. Sci. 24: 795-798 (2008)).

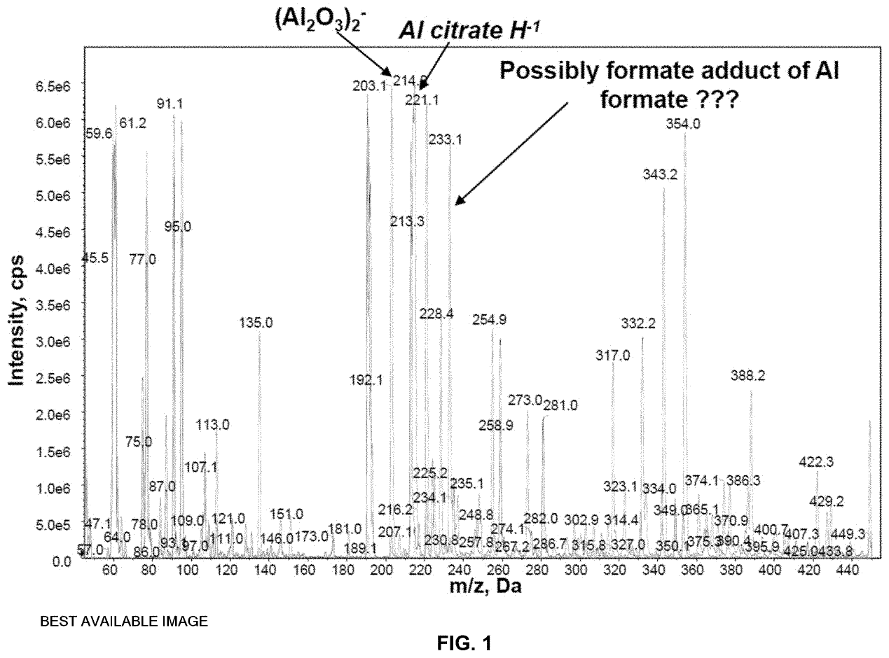

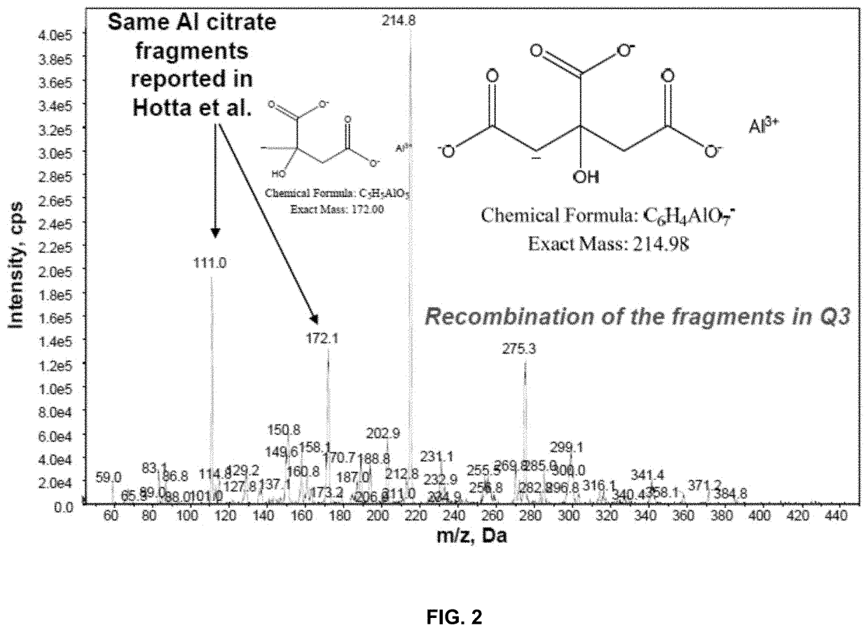

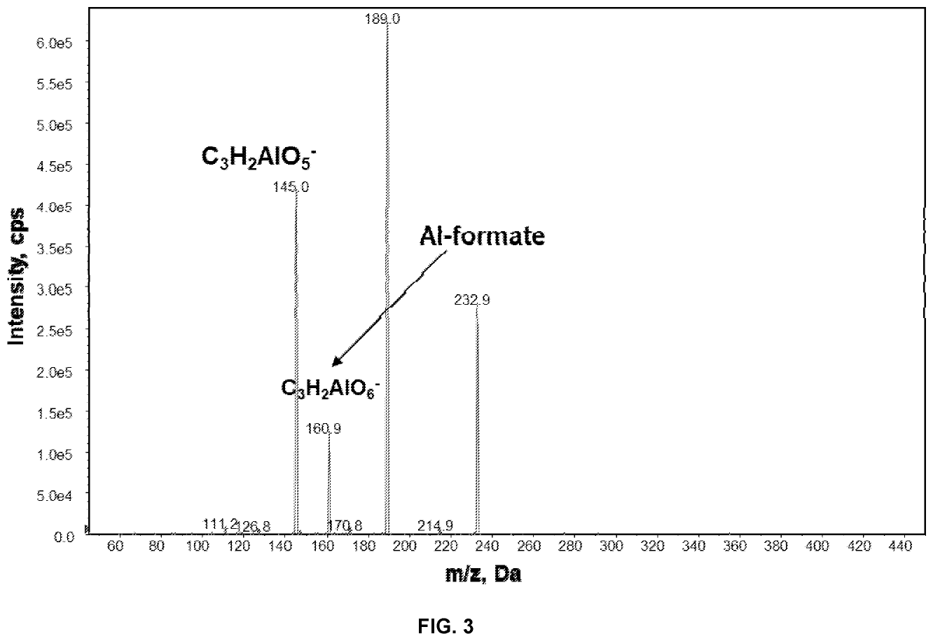

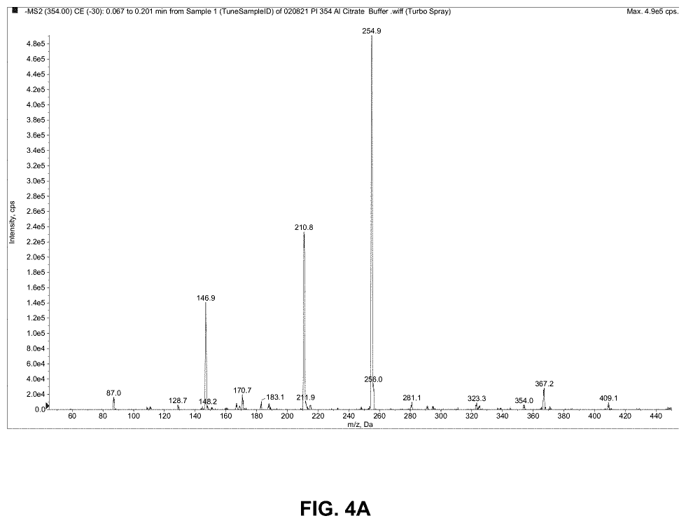

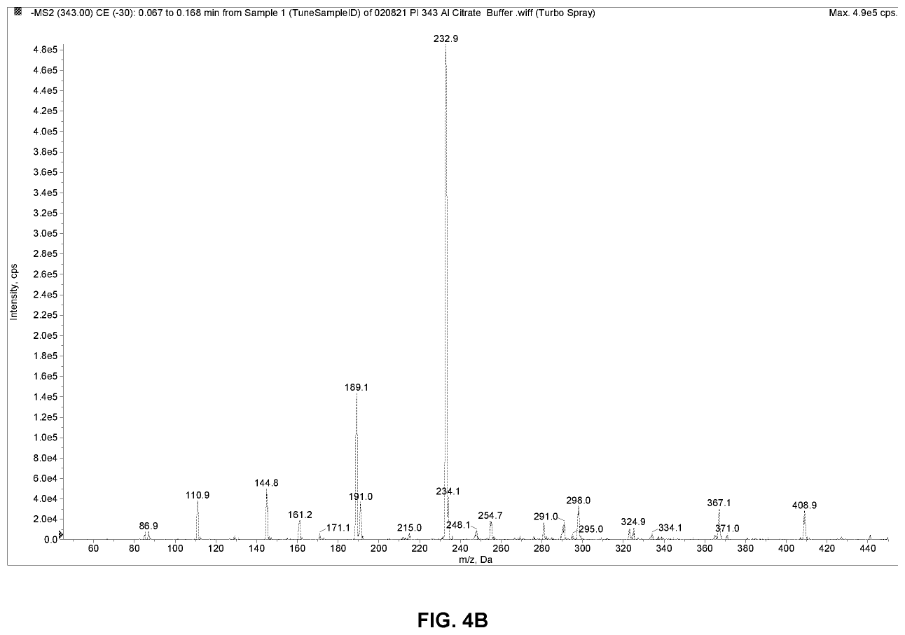

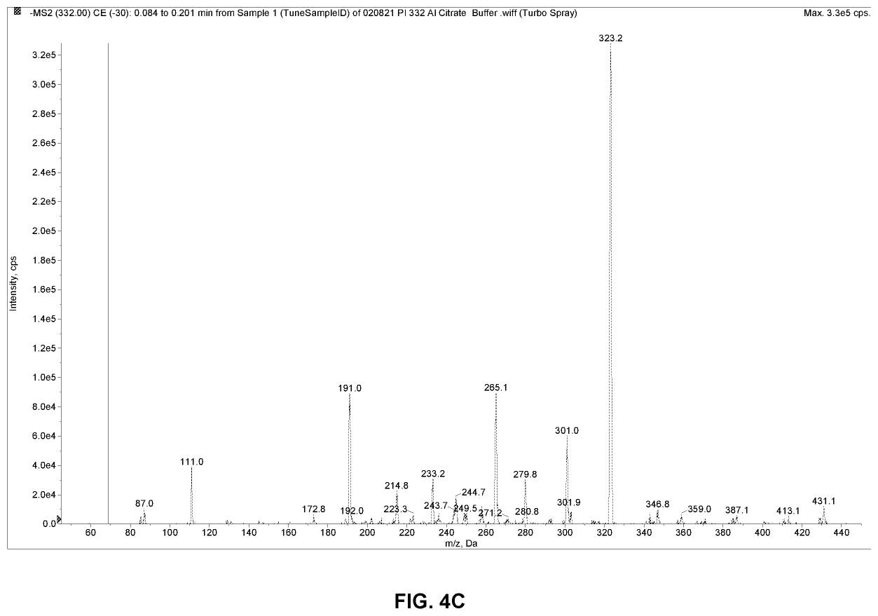

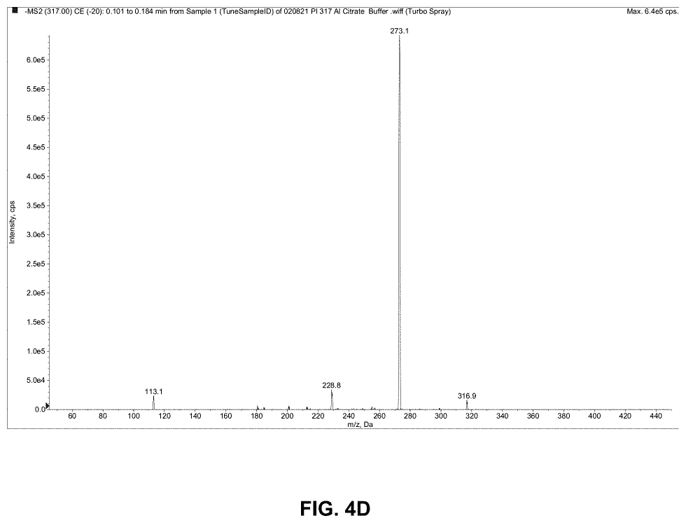

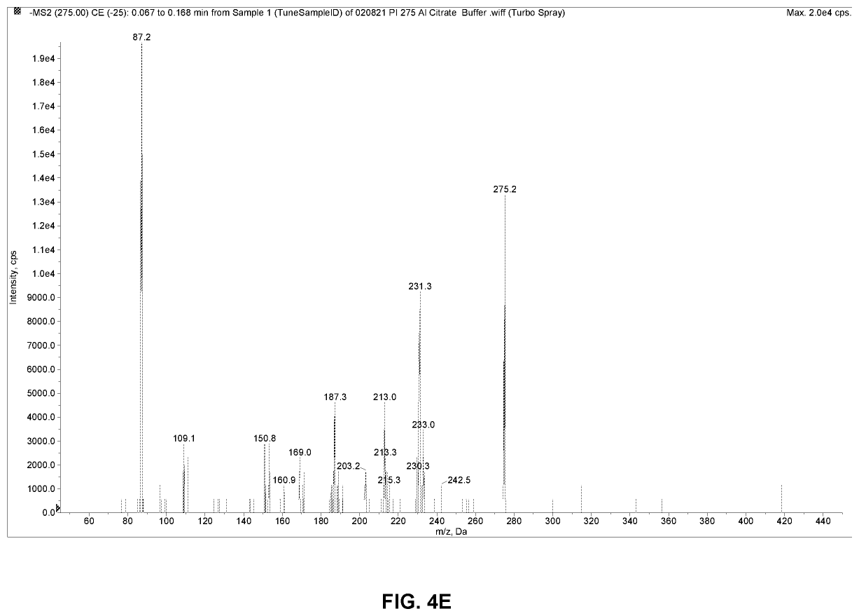

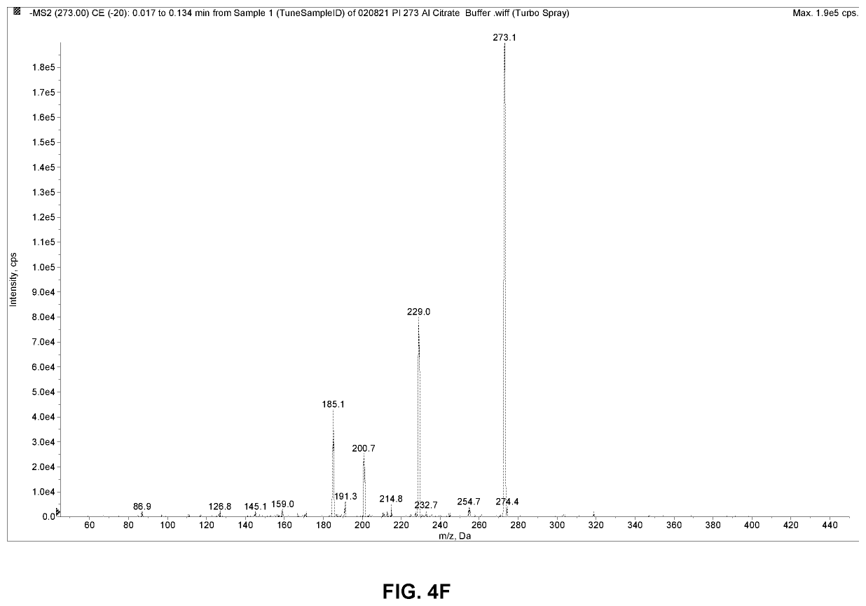

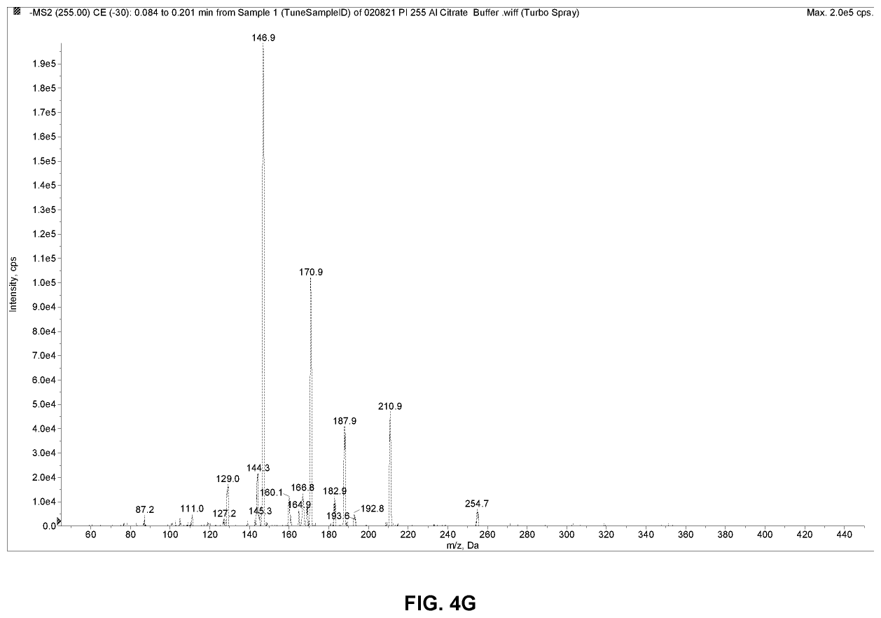

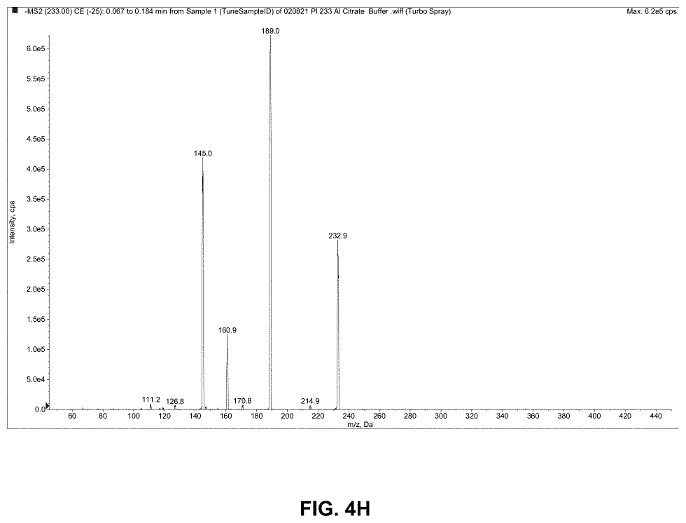

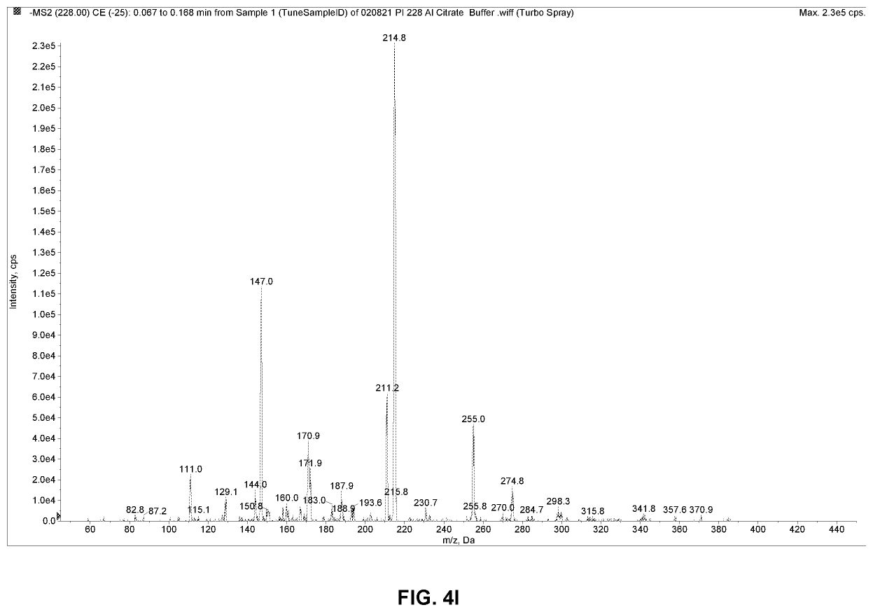

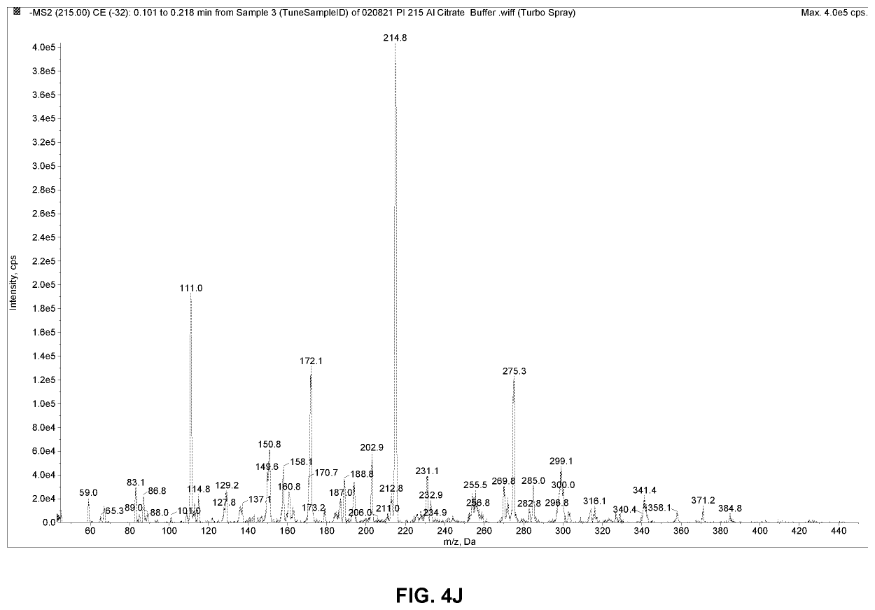

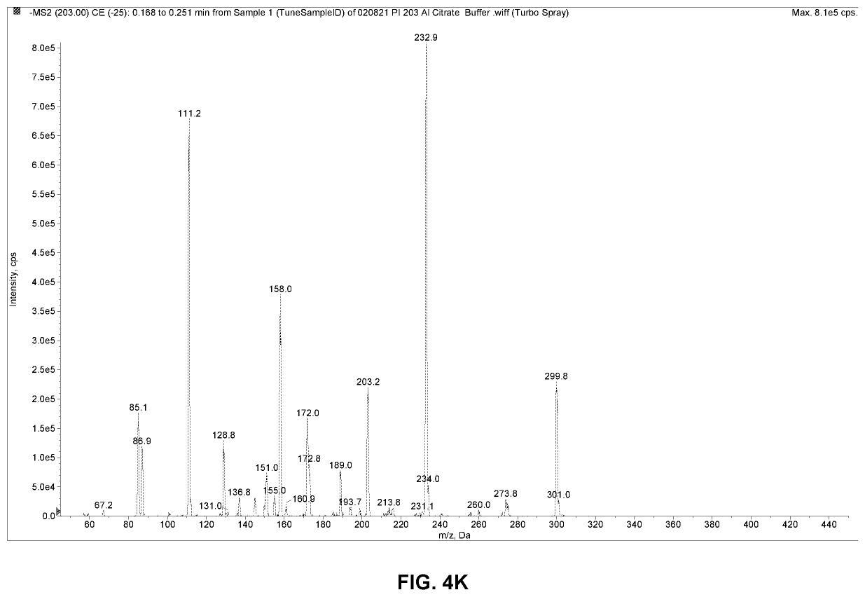

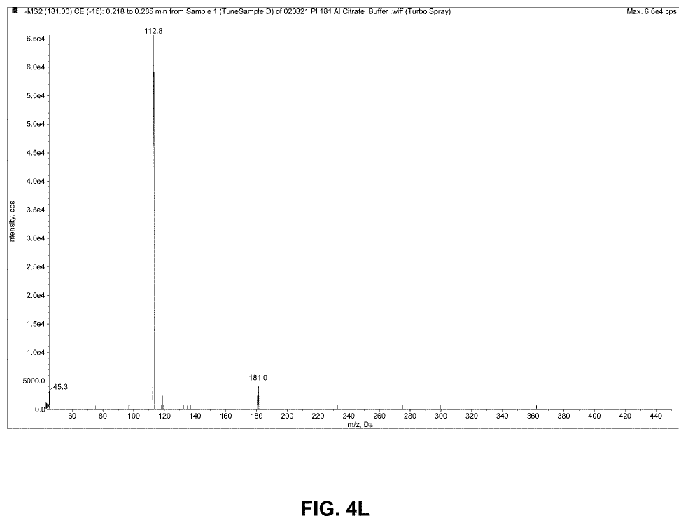

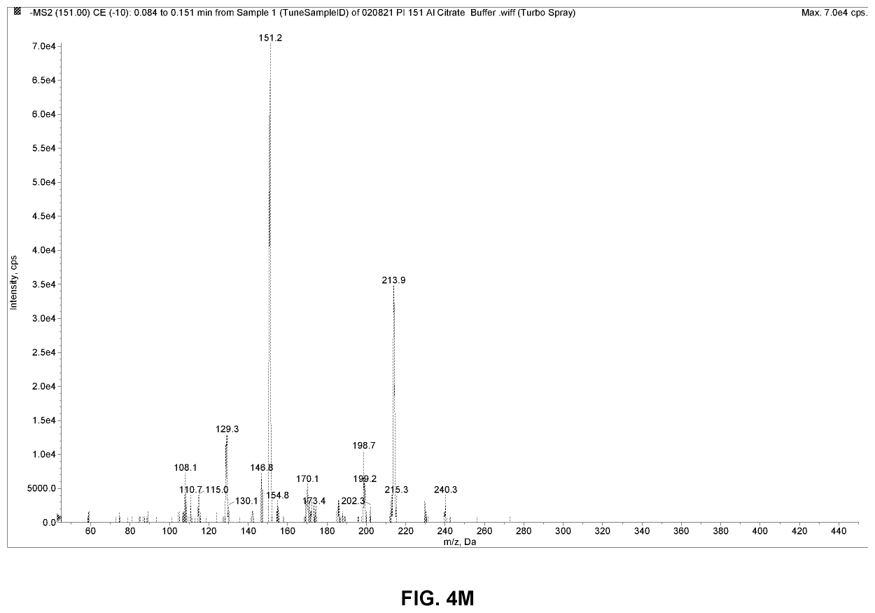

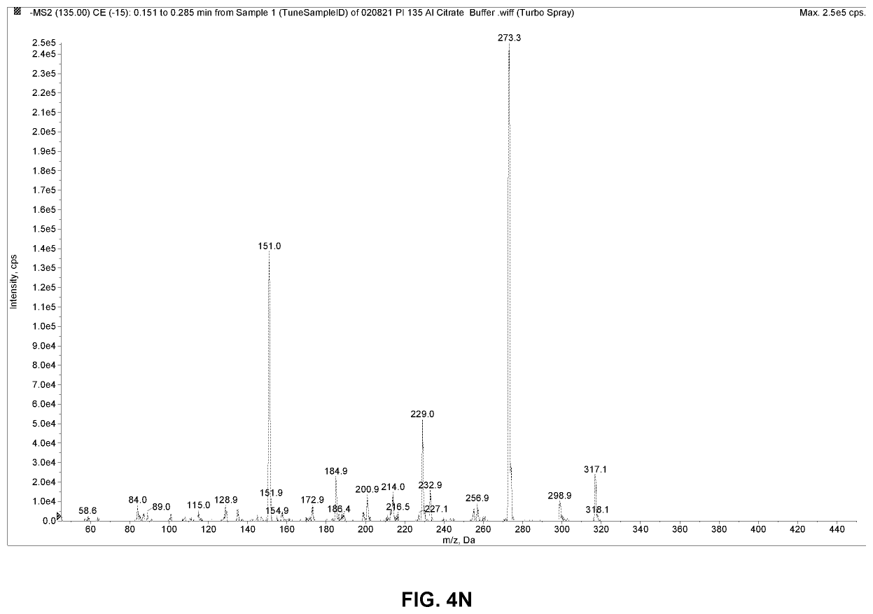

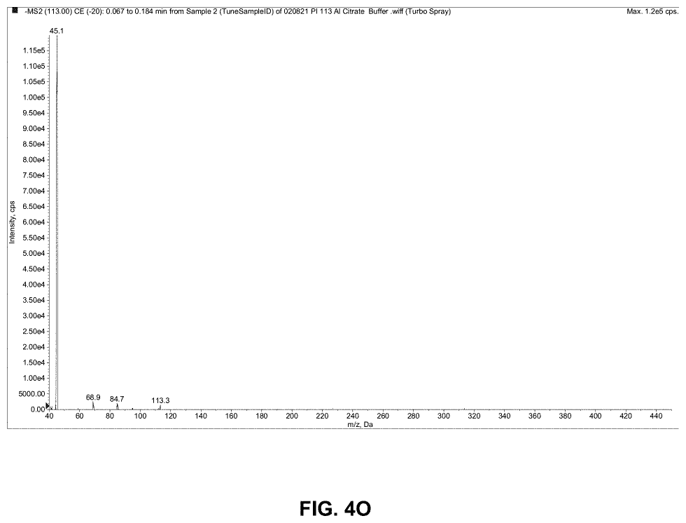

An aliquot of the solution resulting from the reaction between a citrate buffer solution and activated aluminum was further dissolved in methanol/water and electrosprayed. Results are shown in FIGS. 1-3 and FIGS. 4A-4O. The ions corresponding to the presence of aluminum citrate (in both Q1 and product ion mass spectrometry modes) were identified (product ion scan showed the same fragments as previously reported by Hotta et al.), demonstrating successful synthesis of aluminum citrate.

### Example 4: Characterization of a Reaction of Activated Aluminum with an Aqueous Oxalate Buffer

Activated aluminum was reacted with an aqueous oxalate buffer to produce soluble aluminum oxalate and analyzed in a manner similar to Example 3.

A standard solution of aluminum oxalate was prepared using a commercially high purity aluminum oxalate solid, which was dissolved in a methanol/water solution and electrosprayed. Aluminum oxalate ions were identified via primary mass spectrometry (Q1 mode), as previously reported in Al Sadat and Archer 2016 Jul. 20; 2(7):e1600968.

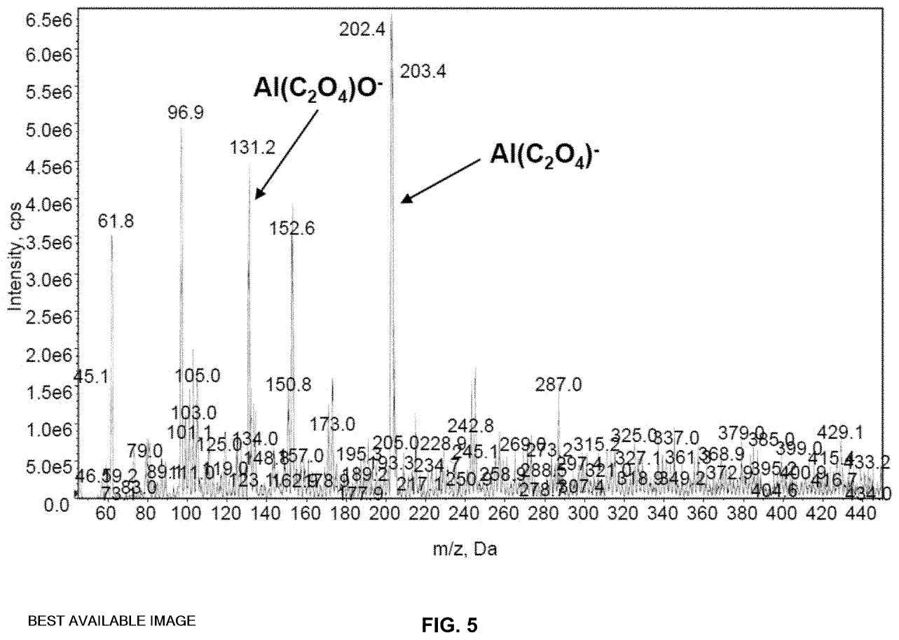

An aliquot of the solution resulting from the reaction between an oxalate buffer solution and activated aluminum was further dissolved in methanol/water and electrosprayed. Results are shown in FIG. 5. The ions corresponding to the presence of aluminum oxalate (in Q1) were identified, demonstrating successful synthesis of aluminum oxalate.

### Example 5: Advantages of the Methods Disclosed Herein

The present invention offers certain advantages of prior methods, including, but not limited to:

The use of activated aluminum metal as a reactive precursor in the methods disclosed herein offers convenience over prior methods of preparing aluminum salts. Typically, elemental aluminum (Al⁽⁰⁾), must be generated using strong acids or bases in situ, making the overall process complex and expensive. Activated aluminum, however, is a solid, shelf stable material that is typically stored under dry conditions (i.e. in a glass jar), can be handled with minimal risk, and does not require a preliminary dissolution step prior to synthetic reaction (i.e. the activated aluminum reacts directly from the solid phase).

Additionally, the ability to use activated aluminum as a singular aluminum source for the synthesis of a variety of different end-product aluminum salts is advantageous in avoiding the need to procure or synthesize additional reactant/precursor aluminum salts. Rather, direct injection of Al⁺³ and Al⁺¹ species into a solution allows for direct reaction with any anions previously dissolved in that solution.

Further, in some instances, the methods disclosed herein produce the desired product without impacting solution pH in an undesired way.

Moreover, the methods disclosed herein allow for the use less expensive and/or less caustic/dangerous reactants when carrying out a reaction to produce a desired aluminum salt (in a non-limiting example, aluminum sulfate can be prepared using the methods disclosed herein using less expensive and less caustic sulfate salts and activated aluminum as starting materials, circumventing the use of sulfuric acid).

We claim:

1. A method for preparing an aluminum salt comprising:
(a) reacting activated aluminum metal (Al⁽⁰⁾) with an anion donor in an aqueous solution;
(b) producing an aluminum salt and one or more byproducts of the reaction; and
(c) optionally separating the aluminum salt from the byproducts.

2. The method of claim 1, wherein the activated aluminum metal is solid or slurried activated aluminum metal.

3. The method of claim 1, wherein the anion donor is a soluble acid, a soluble base, or a soluble salt.

4. The method of claim 3, wherein the anion donor is a soluble acid.

5. The method of claim 4, wherein the soluble acid is selected from the group consisting of citric acid, oxalic acid, nitric acid, sulfuric acid, phosphoric acid, hydrogen chloride, and hydrogen fluoride.

6. The method of claim 4, wherein the hydrogen gas (H₂) byproduct of the reaction is allowed to bubble out of solution.

7. The method of claim 1, wherein wherein Al⁽⁰⁾ is reacted with citric acid (H₃(C₆H₅O₇)) to form aluminum citrate (Al(C₆H₅O₇)) and H₂.

8. The method of claim 1, wherein Al⁽⁰⁾ is reacted with oxalic acid (HO₂C—CO₂H) to form aluminum oxalate (Al₂(C₂O₄)³) and H₂.

9. The method of claim 1, wherein Al⁽⁰⁾ is reacted with nitric acid (HNO₃) to form aluminum nitrate (Al(NO₃)₃) and H₂.

10. The method of claim 1, wherein Al⁽⁰⁾ is reacted with hydrochloric acid (HCl) to form aluminum chloride (AlCl₃) and H₂.

11. The method of claim 1, wherein Al⁽⁰⁾ is reacted with phosphoric acid (H₃PO₄) to form aluminum phosphate (AlPO₄) and H₂.

12. The method of claim 4, further comprising collecting the hydrogen gas (H₂) byproduct of the reaction.

13. The method of claim 3, wherein the anion donor is a soluble base.

14. The method of claim 13, wherein the soluble base is tribasic sodium citrate, and the reaction forms aluminum citrate and sodium hydroxide byproduct.

15. An aluminum salt prepared by the method of claim 1.
